(19) United States
(12) Patent Application Publication
(10) Pub. No.: US 2006/0265030 A1
(43) Pub. Date: Nov. 23, 2006

(54) System and method for photodynamic cell therapy

(75) Inventor: David H. McDaniel, Virginia Beach, VA (US)

Correspondence Address: MORRISON & FOERSTER LLP, 1650 TYSONS BOULEVARD, SUITE 300, MCLEAN, VA, 22102, US

(73) Assignee: Light BioScience, LLC, Virginia Beach, VA (US)

(21) Appl. No.: 11/272,042

(22) Filed: Nov. 14, 2005

Related U.S. Application Data
(60) Provisional application No. 60/627,110, filed on Nov. 12, 2004.

(51) Int. Cl. A61N 5/06

(52) U.S. Cl. 607/88

(57) ABSTRACT

The invention can be characterized as method for stimulating or inhibiting gene expression by photomodulating living cells using a source of narrowband multichromatic electromagnetic radiation. The cells may include, among others, nerve cells, skin cells, retinal cells, heart cells, stem cells, brain cells, cells found in human organs, cells found in hair follicles, and cells found in the human eye or retina. Photomodulation may be enhanced using topically or orally administered compositions. The source of narrowband multichromatic electromagnetic radiation may include at least one light emitting diode (LED) that can emit radiation having a wavelength of from about 300 nm to about 1600 nm.

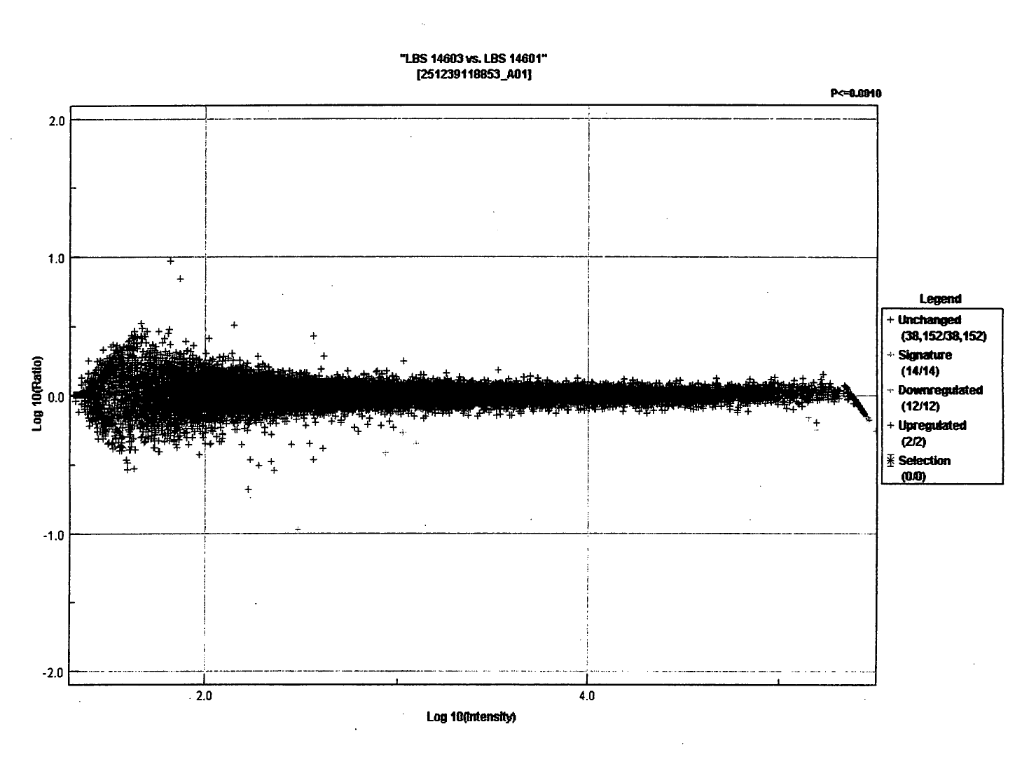

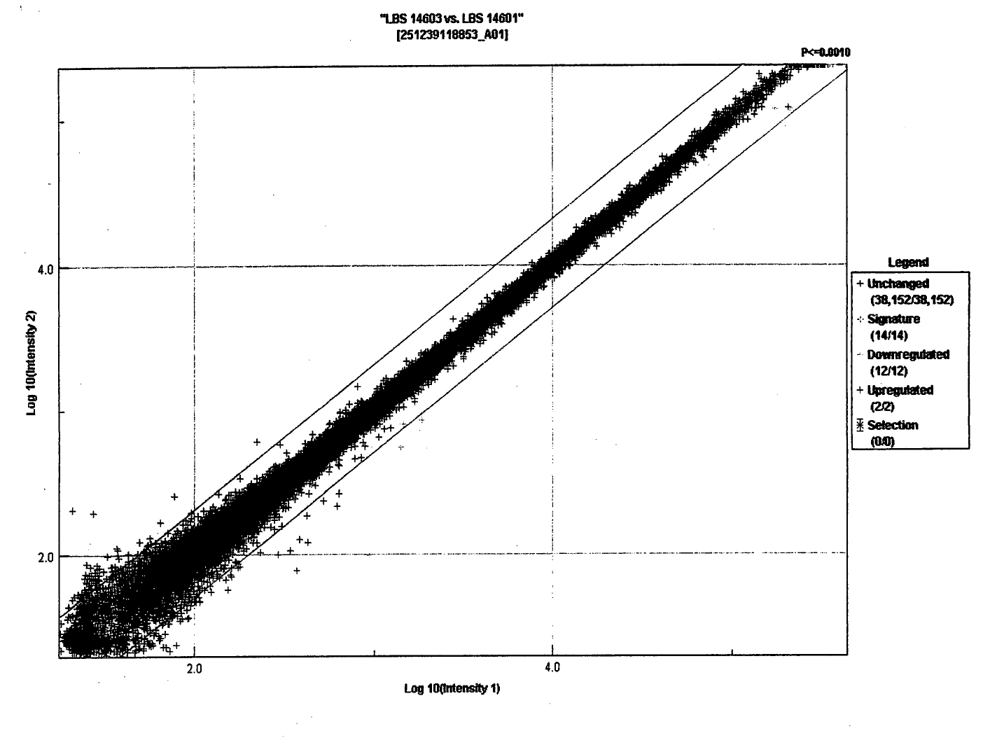

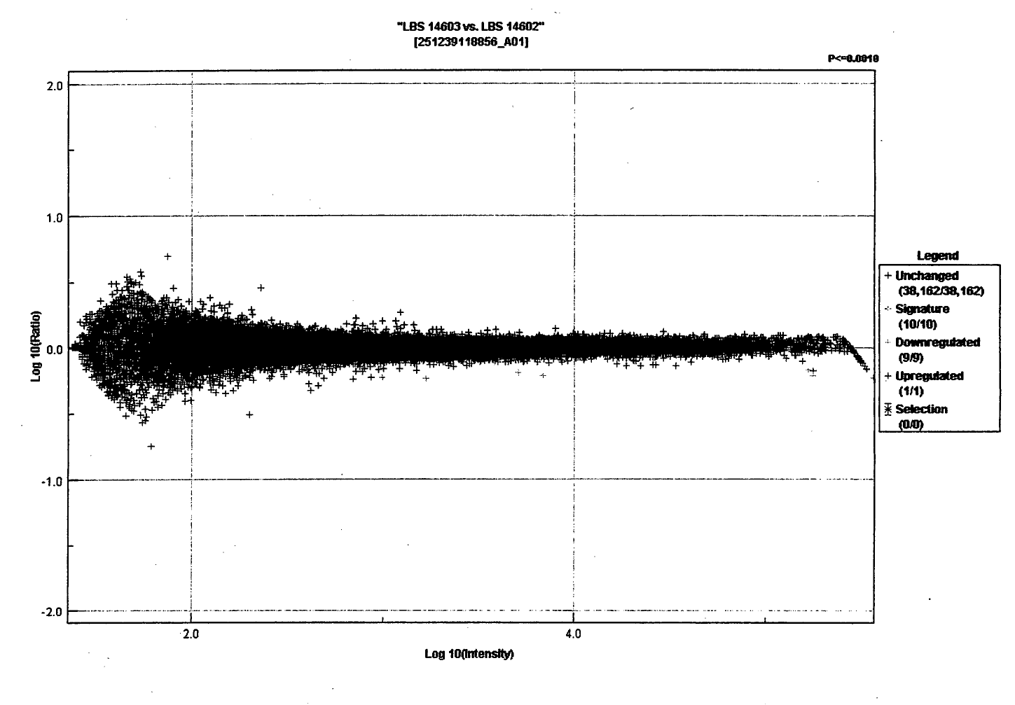

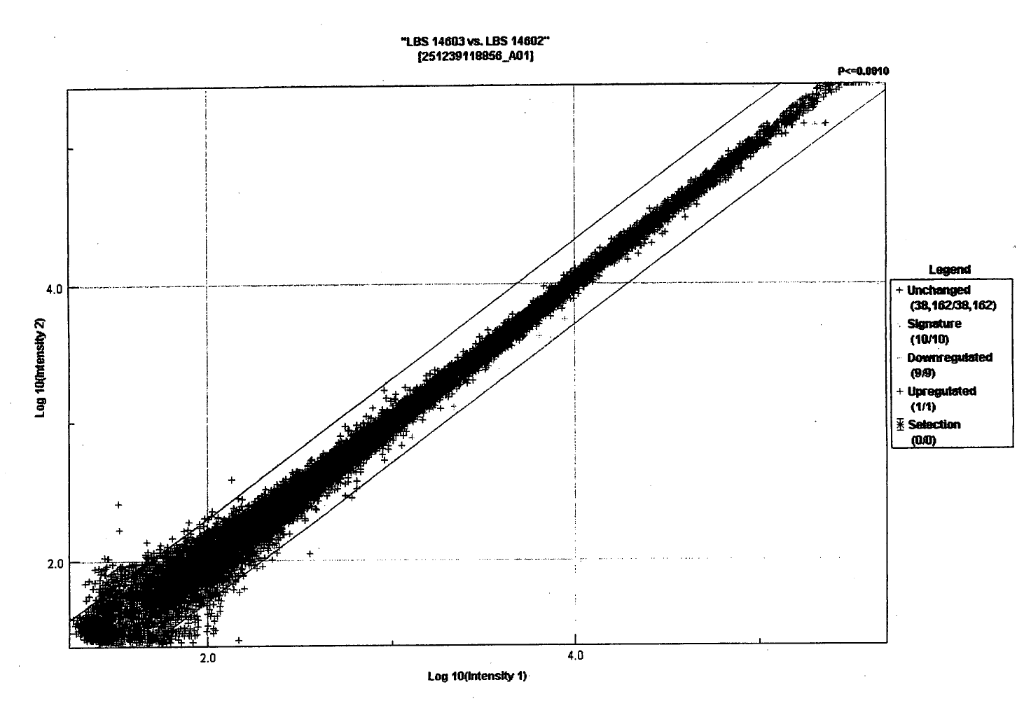

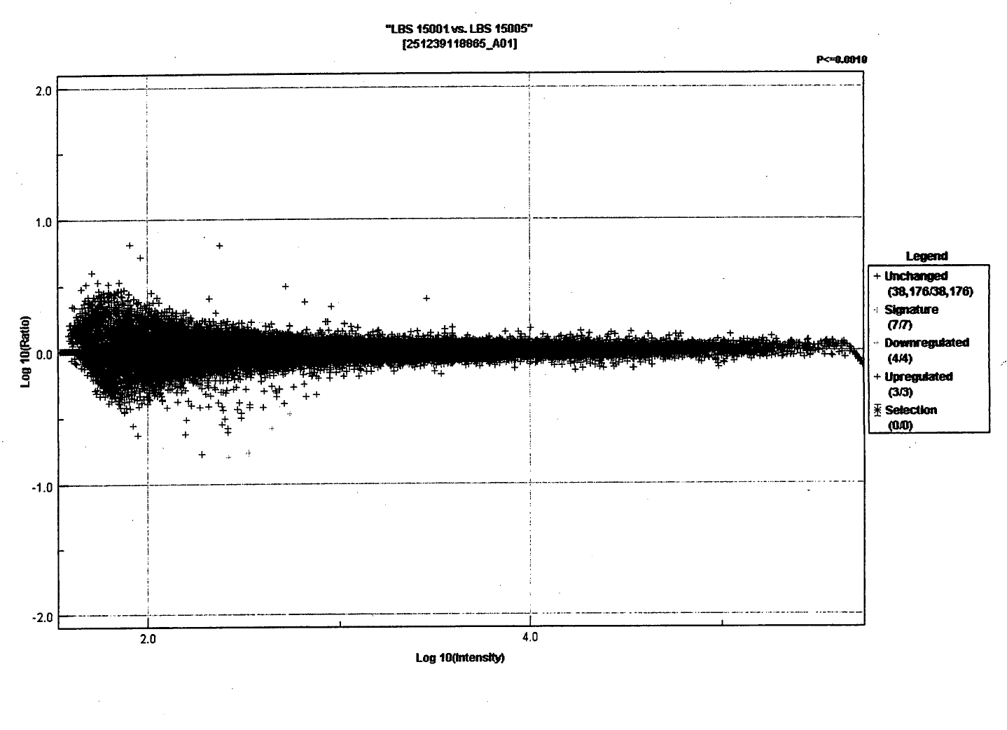

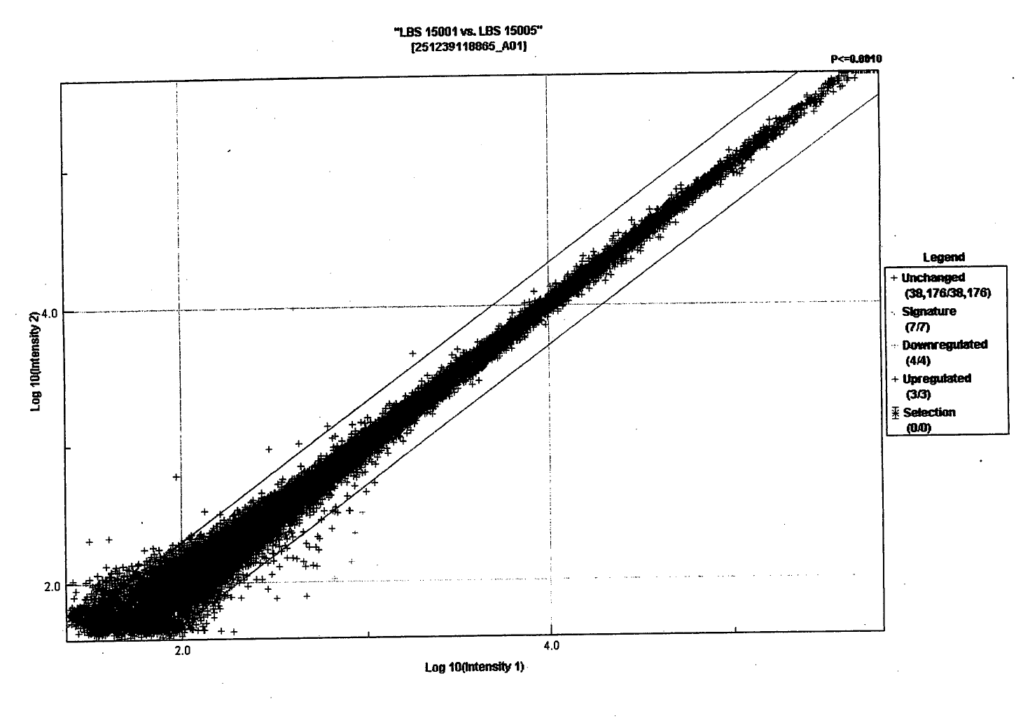

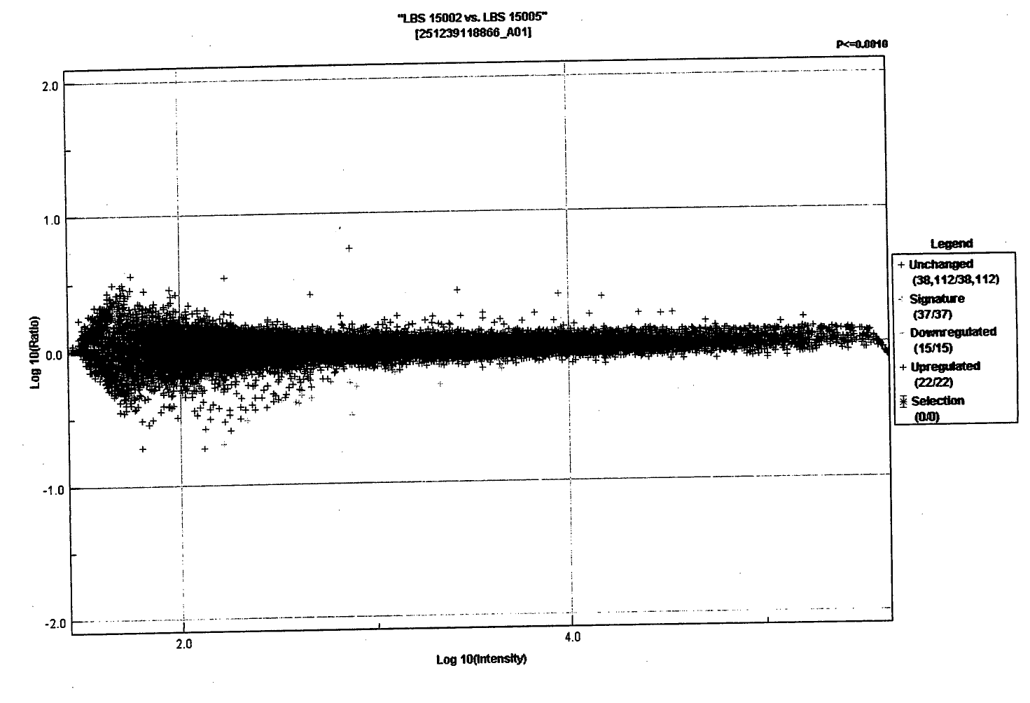

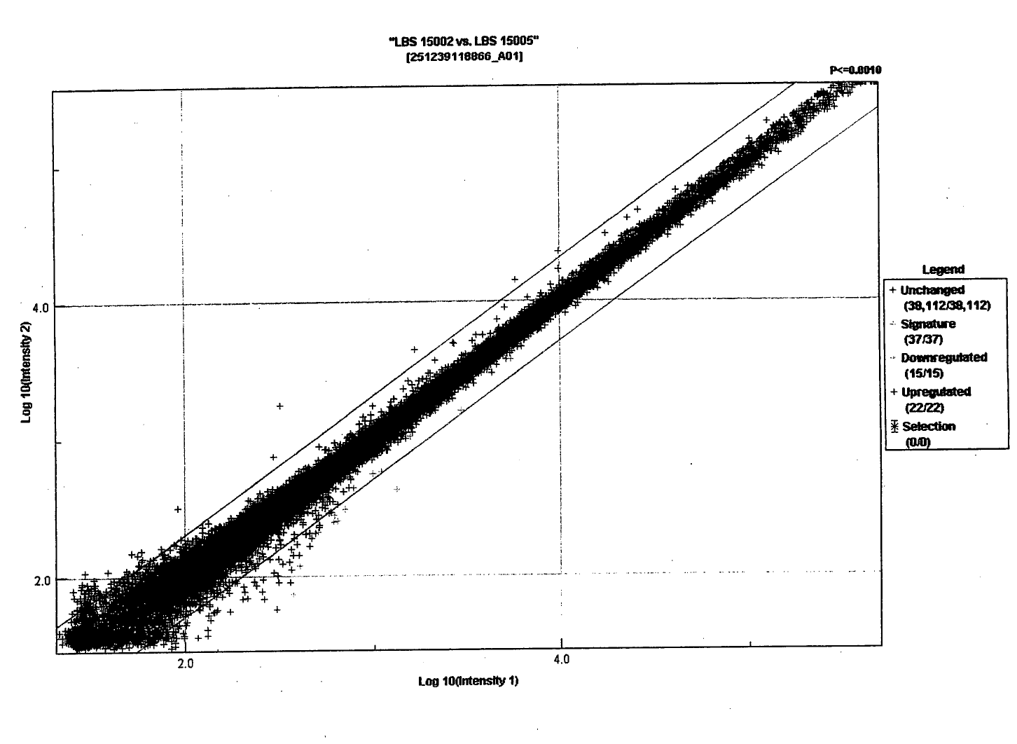

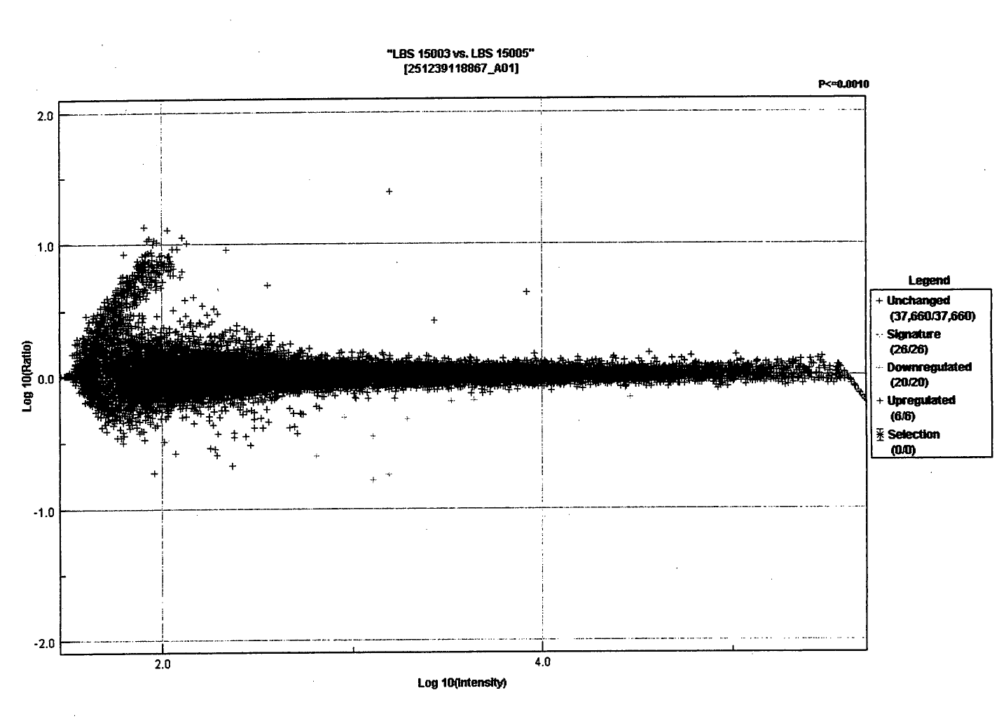

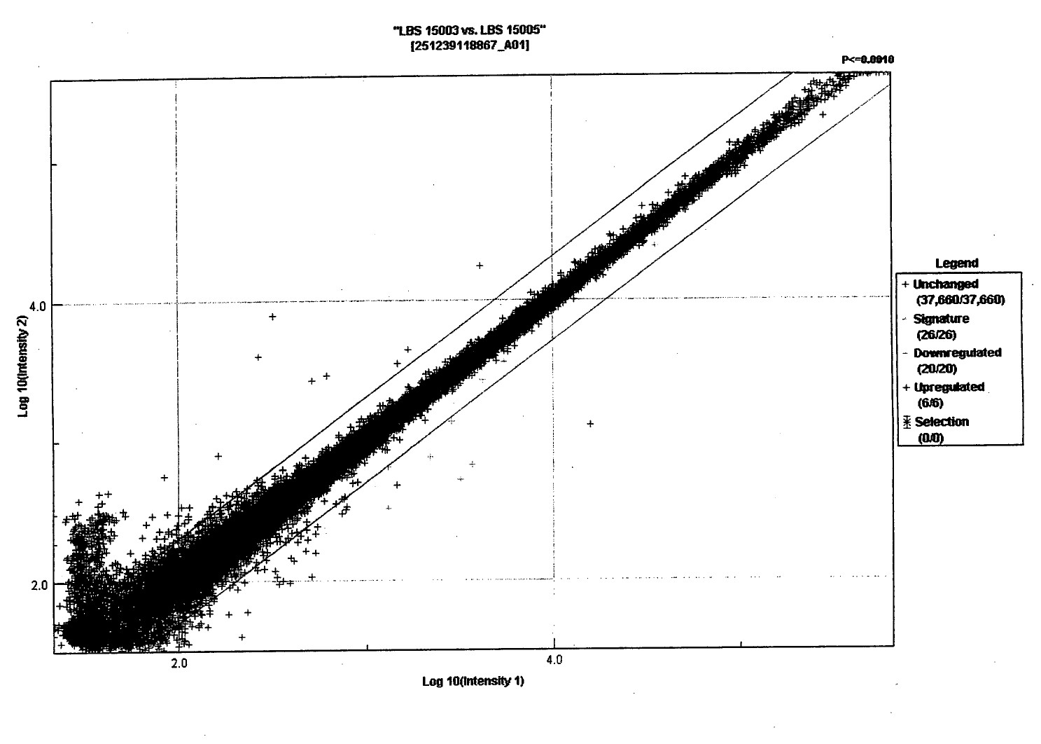

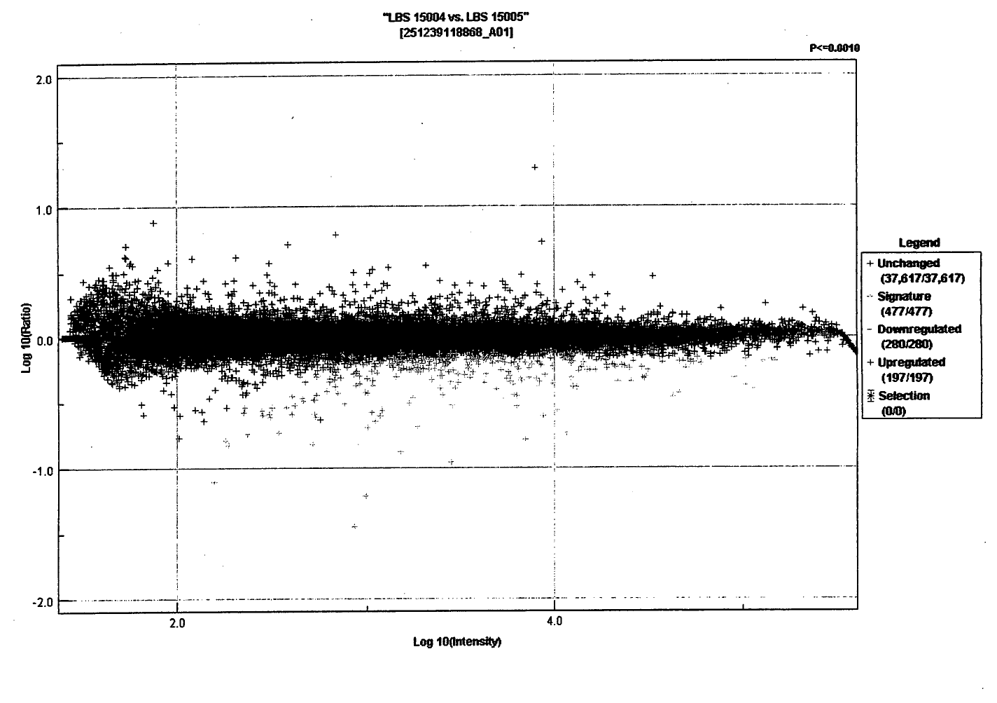

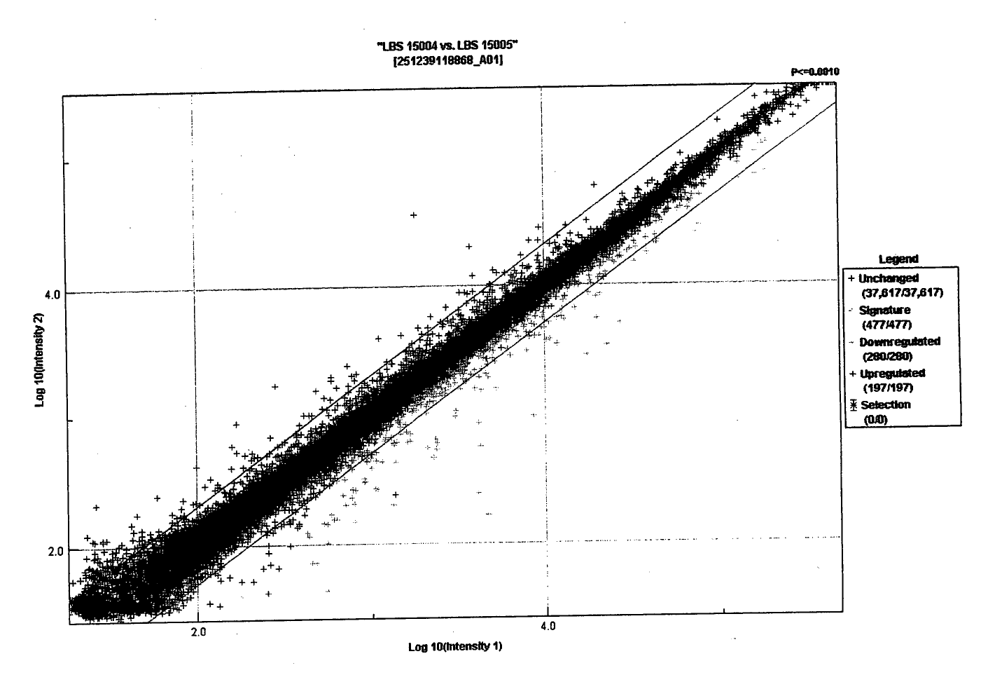

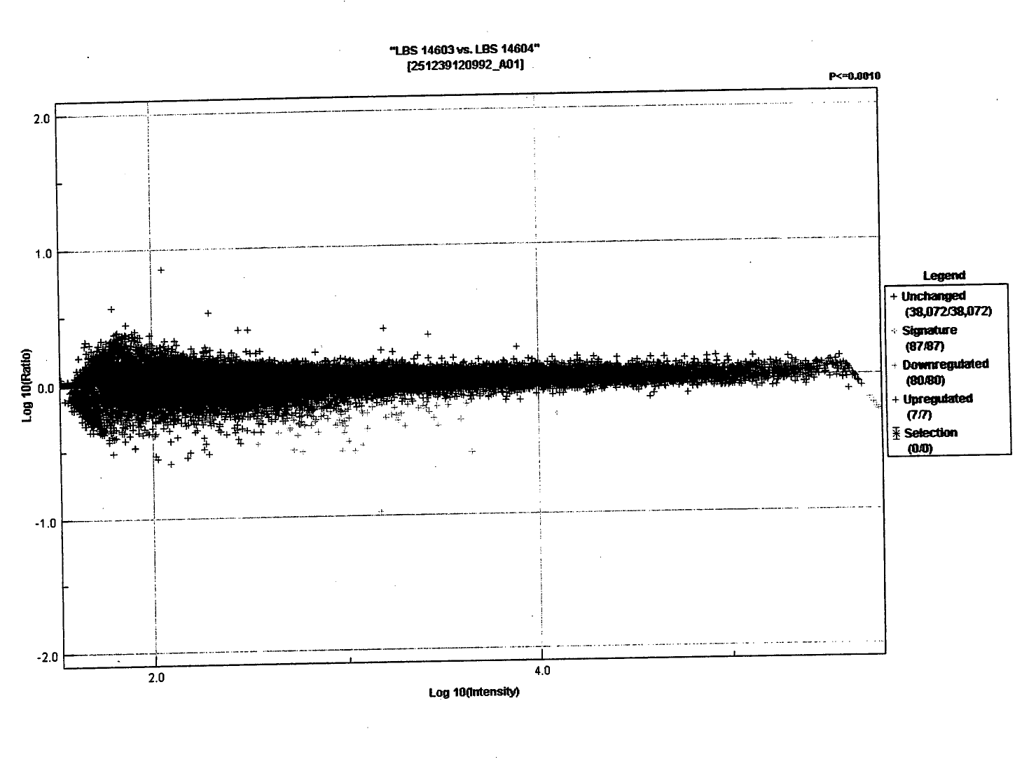

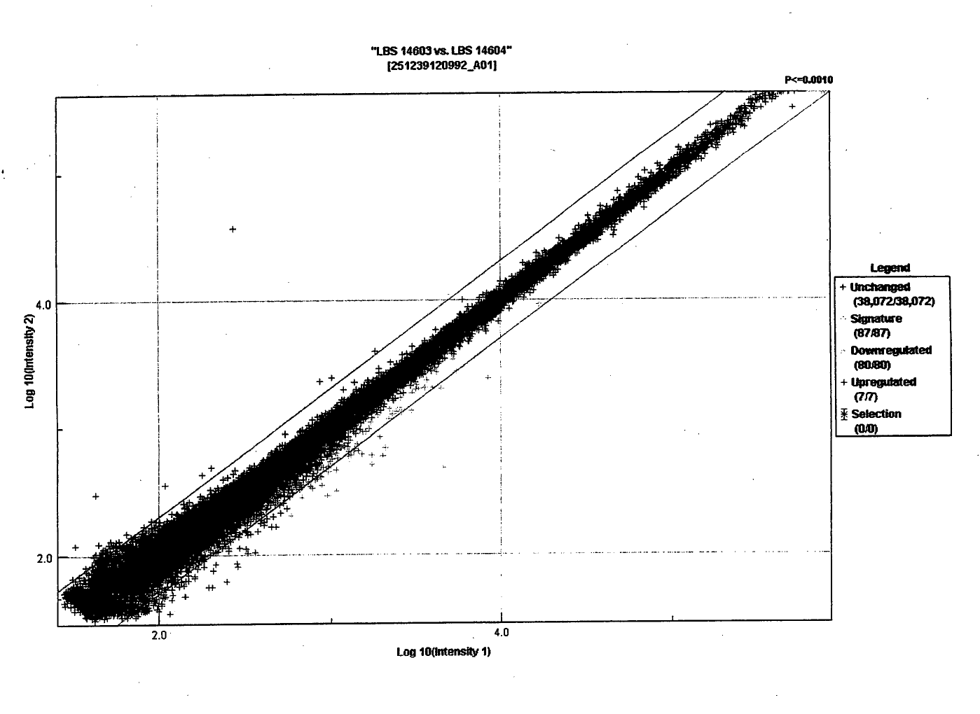

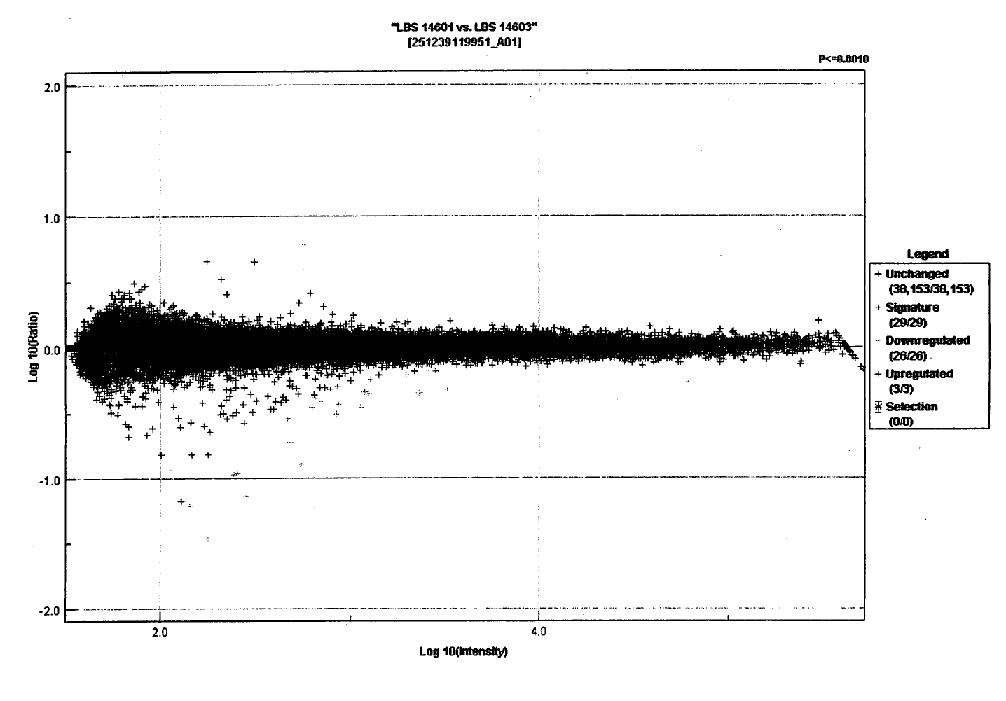

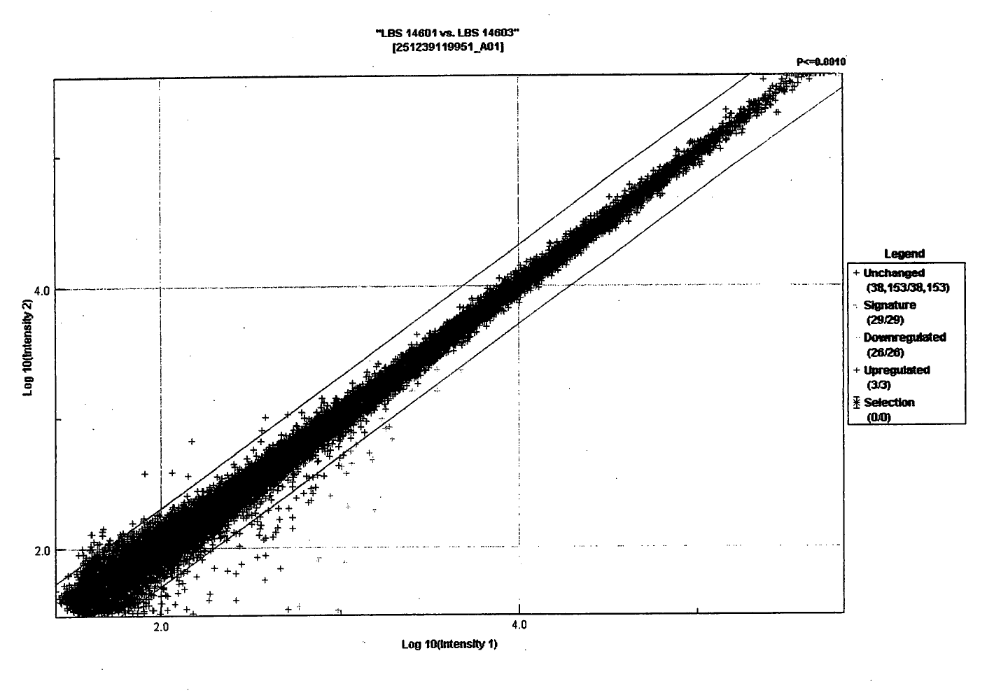

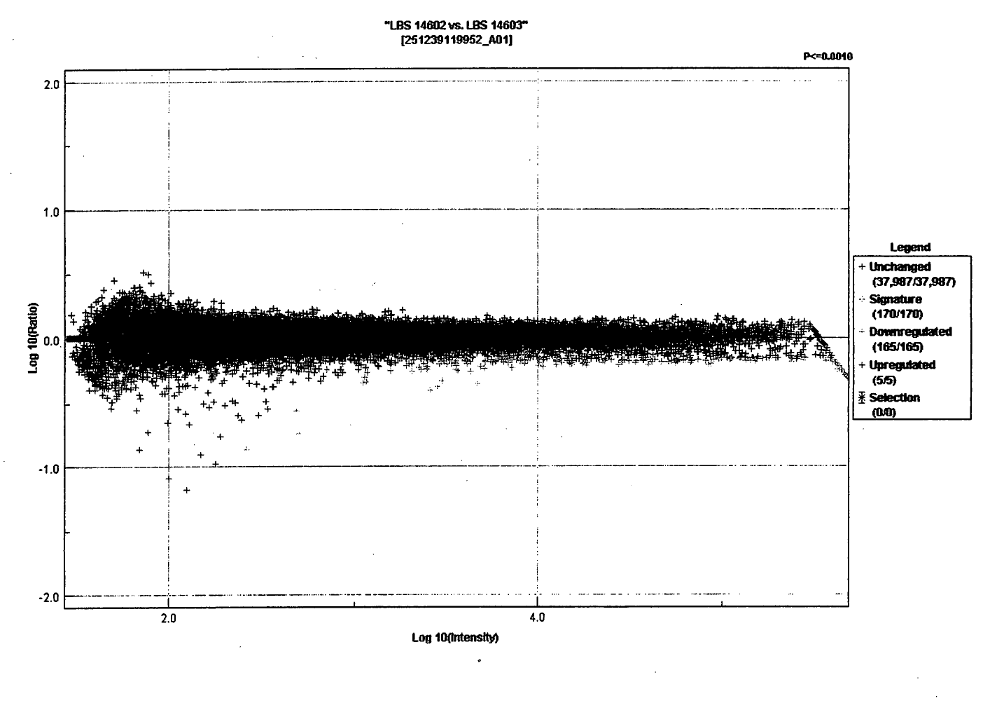

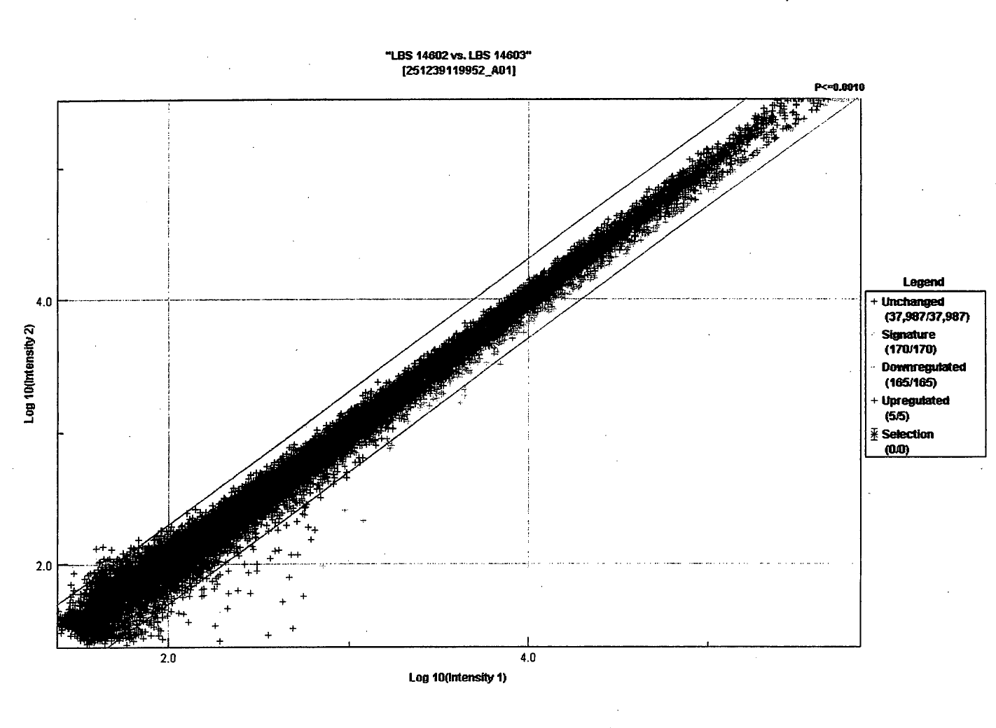

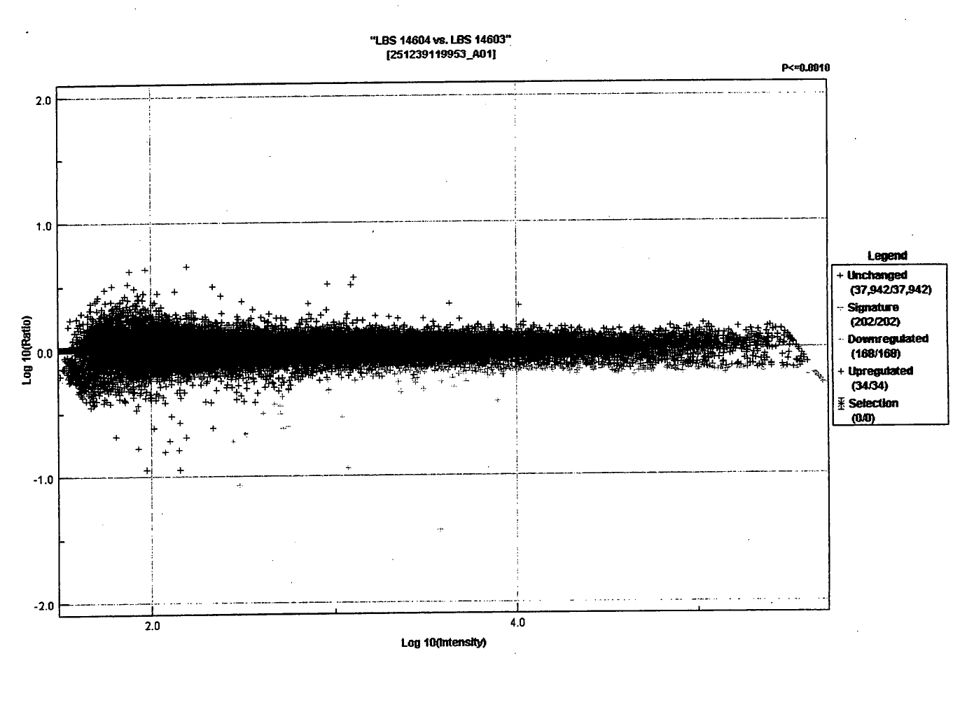

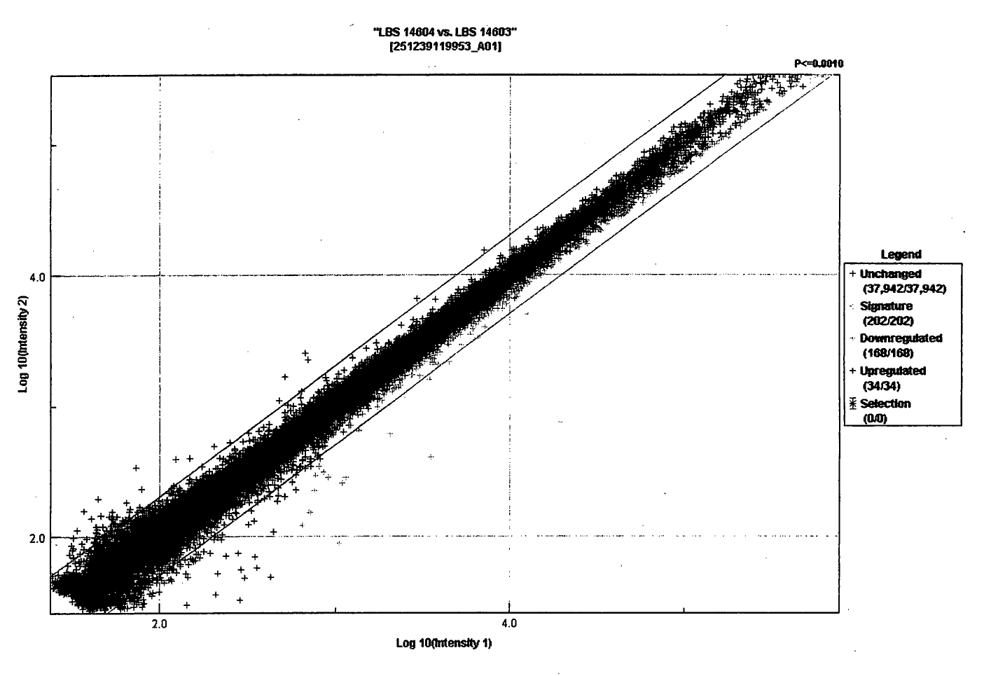

### BACKGROUND OF THE INVENTION

1. Field of the Invention

This invention relates to method and devices for the photodynamic regulation of cell proliferation and gene expression of normal, engineered, autologous, donated, transplanted, apoptotic, necrotic, and other cells which have properties that may be beneficially or detrimentally affected. The cells might include damaged, suboptimally functioning, tumorous, cancerous, mutated or other altered cells existing in or out of the host body, in a laboratory, etc. In particular, the invention relates to photovitalization, photomodulation, photoregulation, and other light-based treatments for cells. In an illustrative embodiment, the invention may be configured to alter normal cell activity, revitalize apoptotic cells, and restore activity to necrotic cells. The light sources may include wideband, narrowband, and other sources of electromagnetic radiation in both the visible and non-visible portions of the spectrum, including electrical stimulation.

The invention further relates to methods and systems for photoregulating and photomodulating the energy production, electron transport, function, and behavior of non gene-based cells such as mitochondria. Such systems employ direct photonic interaction.

2. Background of the Invention

Traditionally, light-based therapies have employed high-intensity, monochromatic sources for performing various treatments on mammalian tissue. More recently, low-intensity, narrowband light sources have been found to have therapeutic effects at the cellular level. For example, it has been found that non-coherent sources of near infrared radiation may protect human dermal fibroblasts from solar ultraviolet toxicity. Further, it has been found that real time RT-PCR indicates a correlation between retinoid-induced apoptosis and NGF-R mRNA levels in cells.

It would be desirable, therefore to be able to influence normal, apoptotic, and even necrotic cells using light sources, to affect the activity of such cells for the purpose of transporting organs (by maintaining cellular activity during transport), photorevitalizaing aging cells, and photorejuventating injured, dying, or dead cells. As well, of particular interest is the photovitalization of apoptotic cells—those which are undergoing pre-programmed cell death. Apoptosis denotes the complex contortions of the membrane and organelles of a cell as it undergoes the process of programmed cell death. During said process, the cell activates an intrinsic suicide program and systematically destroys itself in a controlled manner or by a self-regulated process. The following series of events can be observed:

The cell surface begins to bleb and expresses pro-phagocytic signals. The whole apoptotic cell then fragments into membrane-bound vesicles that are rapidly and neatly disposed of by phagocytosis, so that there is minimal damage to the surrounding tissue. The cell then separates from its neighbors. The nucleus also goes through a characteristic pattern of morphological changes as it commits genetic suicide. The chromatin condenses and is specifically cleaved to fragments of DNA.

Further, U.S. Pat. No. 6,723,798 teaches therapeutic treatment methods and compositions and devices for maintaining neural pathways in a mammal, including enhancing survival of neurons at risk of dying, inducing cellular repair of damaged neurons and neural pathways, and stimulating neurons to maintain their differentiated phenotype. In one embodiment, the invention provides means for stimulating CAM expression in neurons. The invention also provides means for evaluating the status of nerve tissue, including means for detecting and monitoring neuropathies in a mammal. The methods, devices and compositions disclosed therein include a morphogen or morphogen-stimulating agent provided to the mammal in a therapeutically effective concentration. Preferably, however, carrying out a similar function using light-therapy would advantageous due to the reduced cost and less-invasive nature of the treatment.

It would be particularly advantageous to employ light-based means for the photomodulation of apoptotic cells, thereby restoring them to their normal activity state prior to necrosis.

### DESCRIPTION OF THE INVENTION

The invention may be characterized as a system and method for photomodulating cells. The attached pages and charts illustrate the means by which low-intensity light sources, such as light emitting diodes, may cause the rejuvenation of apoptotic and necrotic cells or alter the state of normal cells. For example, the system may provide for the revitalization of skin tissue, hair growth, allow for the preservation of human organs during transport, treating sunburn, thermal and chemical burns and blistering (including those inflicted by chemical warfare agents), scar reduction, hair removal, wrinkle reduction, and the treatment of a wide variety of internal disorders where light may be used to stimulate a desired gene expression. Of particular value are treatments according to the present invention for stem cell disorders, bruising, acute spinal cord trauma, brain stokes, retinal injuries, and heart muscle vitalization and rejuvenation. Such treatments made be used prescriptively, prophylactically, intraoperatively, during post-operative recovery, and other times when it is desirable to affect cell health or behavior.

For application to dermatological disorders, the invention may be applied to a variety of approaches. Although historically, most methods utilize some form of triggering the body's own wound healing mechanism. The more destructive and traumatic methods use chemicals to peel off the stratum cornium epidermis and often a portion of the dermis or they mechanically abraded by sand papering or dermabrating or more recently high-energy thermal lasers have been used to vaporize or coagulate the skin. These methods have a prolonged and painful wounding period and require wound care and patients typically must limit their daily social and business activities during the wound-healing phase. Subsequently the skin undergoes of months or years an on going wound healing and wound remodeling process whereby damage is repaired and new structural proteins in skin are generated. These treatments typically amount to trying to produce a controlled entry to the skin and proving the wound care environment that minimizes the risk of scarring. These methods are notoriously known for producing many problems and sometimes even disfiguring scarring or catastrophic pigment changes in the skin. However, properly performed and with good wound care, many people achieved significant and sometimes dramatic anti-aging effects. Other gentler methods have become more popular in recent years which involve the classic plastic surgery lifting procedures and newer procedures termed non-ablative where the outer stratum cornium and epidermis are not removed or blated from the skin, but are by various means and methods protected and left in tact. Non-ablative methods have typically been thermal in nature and through various means of laser light, intense pulsed light, radio frequency or microwave energy delivery then produced a thermal injury to the dermis. The theory behind these therapies is that this injury will result in a net increase in the desirable structural proteins, while not triggering, worsening, scarring or other complications. Results are occasionally traumatic but have been extremely variable with this therapy. The variability in individuals wound healing repair mechanism and the overall health of their body and skin and many other factors contribute to this variability.

There are various topical agents that have been developed for anti-aging purposes such as Retinoic acid, topical Vitamin C, topical Vitamin E and other antioxidant and other anti-wrinkle creams and lotions. Many of these are well defined. Additional topical compositions, cosmeceuticals, etc. are disclosed in applicant's copending application serial number U.S. Ser. No. 09/899,894, entitled “Method and Apparatus for the Photomodulation of Living Cells”, filed Jun. 29, 2001, which is hereby incorporated by reference in its entirety. Further, methods for enhancing the penetration of such composition into the skin using ultrasound radiation are described in U.S. Pat. No. 6,030,374, and U.S. Pat. No. 6,398,753, each of which is hereby incorporated by reference in its entirety. Use of such compositions for wound treatment, acne reduction, and other dermatological conditions is described in applicant's copending application Ser. No. 09/933,870, filed Aug. 22, 2001, which is also incorporated by reference herein in its entirety. Additional discussion of the related art is described in applications copending application Ser. Nos. 10/119,772, filed Apr. 11, 2002, and 60/461,512, filed Apr. 10, 2003, which are also incorporated by reference herein in their entirety.

The present system contemplates the use of light-based therapy to stimulate gene expression within cells and direct photon stimulation of cells, as described generally in the attached figures. Methods to modulate cell growth or proliferation and gene expression include exposure to electromagnetic radiation in an amount or dose that is sufficient to stimulate the desired effect (e.g. see U.S. Pat. Nos. 6,398,753, 5,837,224, and 6,130,254; and U.S. Patent Application Nos. 2002/0028185, 2001/0053347, 2003/0004556, 2003/0004499, and 2002/0123746, all of which are specifically and entirely incorporated by reference). For example, exposure of skin to LED can stimulate or inhibit the expression of various gene products. These same methods can be used to cause stimulation or inhibition of cell proliferation or differentiation and cell cycle modulation in these cell populations. Further, photomodulation can be used in combination with certain oral agents (for systemic affects) or topical agents (for localized affects) (e.g. vitamin A, retin A, retinol), for a desired effect unachievable with either stimulant used individually.

The types of cells that can be affected include, but are not limited to skin cells (reversal of photoaging), nerve cells (disease prevention and treatment), stem cells (tissue reconstruction), cells of hair follicles (hair growth or inhibition), cells of the immune system including cells intimately involved with the process of inflammation (due to disease, infection, or congenital disorder), wound repair, eye/retina cells, heart cells, brain cells, entire organs, and combinations thereof. Modulation can be achieved by exposing cells to electromagnetic radiation (e.g. photomodulation) such as, preferably, visible light, (e.g. purple, blue, green, yellow, orange, red), infrared radiation, ultraviolet light (UVA, UVB, UVA1, UVA2, or combinations thereof), or combinations of any. Preferred exposure strengths and exposure times are as set forth in the attachments hereto, but may include pulsed exposures, continuous and periodic exposures.

Regulation of gene expression by light in living cells. Photomodulation of gene expression occurs in both nucleus and mitochondria. The following mechanisms are relevant to the use of light to regulate gene expression. 1) Light Capture—photons captured by antennae molecules or receptors; 2) Light Energy Transfer—photon energy is transduced into a signal; 3) Signal Coupling—the signal transduction couples to gene expression; and 4) Gene Expression—cellular activities and cell products regulated by gene expression.

Types of Regulation include: PhotoRegulation, PhotoRejuvenation, PhotoRevitalization, PhotoRegeneration, and PhotoReregulation. Photomodulation is determined by a set of parameters which may be termed the ‘cellular photomodulation code: light intensity (irradiance); spectral quality (spectral wavelength, spectral bandwidth, spectral ratio (ratio of different wavelengths), and polarization

Factors which may be varied to achieve different levels of expression in particular genes or to cause expression in other genes include: light exposure (duration), frequency (if pulsed), time (pulse duration), off time (dark time), total number of pulses, interval between exposures (single/multiple wavelengths). synchrony (simultaneous or sequential).

Inhibition, modulation, quenching my occur by ‘interfering’ light or electromagenetic radiation or other factors which disrupt or modulate normal cell signal transduction. Competing endogenous or exogenous chromophores in living cells or tissue may alter spectral quality or photomodulation process. Photodamage may also occur (which is different than photoinhibition) and may be due to ‘excess’ photon flux or excess total number of photons.

Photophosphorylation is significant in cell transduction process. Reactive center/antenna molecule is the ‘Portal’ connecting the world of physical ‘light’ energy and biological life—this is central concept in the photomodulation of living cells and life processes. Redox state of primary electron acceptor ‘controls’ photomodulation (and photodamage). Maximum effect on gene expression may require photomodulation of more than one receptor (i.e., upstream or downstream reactive center/receptor photomodulation in addition to ‘primary’ receptor).

We claim:

1. A method for stimulating, inhibiting, or regulating gene expression, comprising: exposing a living cell comprising at least one gene to a source of narrowband, multichromatic electromagnetic radiation.

2. The method of claim 1 wherein the source of narrowband, multichromatic electromagnetic radiation comprises at least one light emitting diodes.
